(19) United States
(12) Patent Application Publication
(10) Pub. No.: US 2024/0021285 A1
(43) Pub. Date: Jan. 18, 2024

(54) SYSTEM FOR MEDICAL TREATMENT RECOMMENDATION AND PREDICTIVE MODEL THEREOF

(71) Applicant: OSMIND INC., San Francisco, CA (US)

(72) Inventor: Matthew Jacob Worley, Austin, TX (US)

(73) Assignee: OSMIND INC., San Francisco, CA (US)

(21) Appl. No.: 18/221,867

(22) Filed: Jul. 13, 2023

Related U.S. Application Data
(60) Provisional application No. 63/388,966, filed on Jul. 13, 2022.

(51) Int. Cl. G16H 20/17; G16H 10/20

(52) U.S. Cl. CPC G16H 20/17; G16H 10/20

(57) ABSTRACT

A method for adherence prediction, comprising the steps of isolating a training dataset and a test dataset from the full dataset; splitting the training dataset into training folds and a validation fold; training, over a plurality of trials, one or more models on each of the training folds for each of one or more parameter configurations correlated to said model, each of the models configured to classify a target variable as an indicator of whether a patient will complete a treatment; validating, for each of the one or more models for a given trial of the plurality of trials, a given model of the one or more models on the validation fold; recording classifications scores for each of the one or more models; selecting a model from the one or more models, the selected model having a top classification score; and retraining the selected model on the full dataset.

### CROSS-REFERENCE TO RELATED APPLICATIONS

The present application claims the benefit of U.S. Patent Application No. 63/388,966 for SYSTEM FOR MEDICAL TREATMENT RECOMMENDATION AND PREDICTIVE MODEL THEREOF, filed Jul. 13, 2022, the entire contents of which are incorporated herein by reference in their entirety.

### FIELD OF THE INVENTION

The present disclosure relates to systems and methods for evaluating treatment efficacy. Specifically, the present disclosure relates to systems and method for determining likelihood of IV ketamine treatment adherence.

### Introduction

Currently, clinical depression is one of the most common and costly health problems in the world. However, the most popular evidence-based treatments for depression do not produce lasting benefits in roughly 30% of the patients who receive them.

Recently, intravenous (IV) ketamine has emerged as a viable option for treatment of depression, especially in cases when other treatments have failed. Clinical studies have demonstrated IV ketamine typically produces rapid and large reductions in depression when patients receive a full initial course of treatment, i.e., 4-8 infusions within 28 days (the “induction treatment”). Nevertheless, nearly half of patients who receive IV ketamine treatment do not adhere to the prescribed visit routine for the induction treatment. This is problematic for both clinicians and patients. Data shows that patients who are adherent to the prescribed regimen have better outcomes than those who stop treatment prematurely or do not receive induction treatment. Clinicians lack data-driven tools to know when to offer IV ketamine treatment versus alternative treatments. Often patients invest significant financial resources and time into IV ketamine treatment that ultimately may not work for them. Such an issue is exacerbated because ketamine for depression is typically not covered by health insurance.

In the current state of IV ketamine clinical practice, a clinician typically conducts an initial baseline evaluation or consultation. After this consultation, the clinician and patient must make an informed decision on whether to begin a course of IV ketamine treatment. To make this decision, clinicians may rely on such factors as whether the patient has uncontrolled hypertension or other neurological or cardiac conditions that could be aggravated by elevated blood pressure, which is a common side-effect of ketamine. Liver enzymes that are three times more prevalent than that of normal levels are a relative contraindication. In terms of psychiatric conditions, psychosis is typically the only condition that is contraindicated. Additionally, patients cannot be pregnant or breastfeeding. Cost is often a prohibitive factor and ketamine IV treatment (“KIT”) may be relatively contraindicated if another treatment is available and the patient can get reimbursed for care. An important factor that would incline a clinician to choose KIT over transcranial magnetic stimulation (“TMS”) or esketamine would be the presence of suicidal ideation as KIT is the most rapid acting of these treatments, potentially bringing relief within weeks.

Traditionally, some clinicians may be aware of individual factors linked to IV ketamine outcomes and may make high-level assumptions of treatment success. However, human clinicians lack the decision-making capacity to weigh multiple factors simultaneously and assign the appropriate weight to each individual factor in an algorithmic manner.

It would be desirable to provide a system configured to predict treatment adherence and/or compliance. It would further be desirable to provide a system to evaluate likelihood of treatment adherence in view of patient characteristics extracted before prescription of said treatment. It would yet be further desirable to perform such a prognostication utilizing standard patient intake data.

### SUMMARY

In an aspect, a computer-implemented method for treatment adherence prediction, may comprise the steps of receiving a full dataset; isolating a training dataset and a test dataset from the full dataset; splitting the training dataset into one or more training folds and a validation fold; and training, over a plurality of trials, one or more models on each of the one or more training folds for each of one or more parameter configurations correlated to said one or more models, each of the one or more models configured to classify a target variable, wherein the target variable is an indicator of whether a patient will complete a treatment. In a further embodiment, the method may comprise the steps of validating, for each of the one or more models for a given trial of the plurality of trials, a given model of the one or more models on the validation fold; comparing a training score with a validation score, wherein the training score is based on the training of the one or more models over the one or more training folds, and wherein the validation score is based on the validating of the given model on the validation fold; and recording classifications scores for each of the one or more models, the classifications scores based on the training score and the validation score for each of the one or more models. In yet a further embodiment, the method may comprise the steps of selecting a selected model from the one or more models, the selected model having a top classification score; retraining the selected model on the full dataset; and generating adherence predictions for the test dataset.

In an embodiment, the treatment comprises intravenous (IV) ketamine infusions. In one embodiment, the completeness of the treatment is defined as the patient completing at least four IV ketamine infusions within twenty-eight days from an intake evaluation.

The one or more parameter configurations may be based on a list of settings associated with each of the one or more models. In an embodiment, the one or more models are selected from a group of classifier types comprising Bayesian linear models, hierarchical models, naive Bayes classifiers, and kernel-based methods. At least one of the one or more models may be an ensemble model.

In an embodiment, at least the training dataset comprises a plurality of variables, wherein each of the one or more models is configured to classify a target variable based on each of the plurality of variables. The plurality of variables may comprise one or more continuous variables and one or more binary variables, wherein each of the one or more continuous variables is an integer or real number, and wherein each of the one or more binary variables is encoded as 1 if true, −1 if false, and 0 if missing. As a non-limiting example, the plurality of variables comprises a normalized population density of a resident zip code, a normalized median income of a resident zip code, a normalized median home price of a resident zip code, a normalized number of total ICD-10 diagnoses, and a normalized number of ICD-10 diagnoses considered as psychiatric conditions. As a non-limiting example, the plurality of variables comprises a normalized number of prior patients treated by a clinic with KIT, a normalized proportion of prior patients at the clinic that met a threshold for adherence to KIT, the patient's age at first infusion, the patient's BMI, and a normalized number of days patient had been associated with the clinic prior to their first KIT treatment. The plurality of variables may comprise a normalized GAD7 composite score and a normalized PHQ9 composite score. In an embodiment, the plurality of variables comprises a GAD-7 Item 1 Score, a GAD-7 Item 2 Score, a GAD-7 Item 3 Score, a GAD-7 Item 4 Score, a GAD-7 Item 5 Score, a GAD-7 Item 6 Score, a GAD-7 Item 7 Score, a PHQ-9 Item 1 Score, a PHQ-9 Item 2 Score, a PHQ-9 Item 3 Score, a PHQ-9 Item 4 Score, a PHQ-9 Item 5 Score, a PHQ-9 Item 6 Score, a PHQ-9 Item 7 Score, a PHQ-9 Item 8 Score, and a PHQ-9 Item 9 Score. In a further embodiment, the one or more continuous variables comprises sex, relationship status, completion status of intake form, mood disorder diagnosis, anxiety disorder diagnosis, attention disorder diagnosis, pre-visit status, and provider physician status. In an embodiment, if the sex is male, said variable value is 1, if relationship status is positive, said variable value is 1, if the completion status of intake form is completed, said variable value is 1, if mood disorder diagnosis is positive, said variable value is 1, if anxiety disorder diagnosis is positive, said variable value is 1, if attention disorder diagnosis is positive, said variable value is 1, if pre-visit status is positive, said variable value is 1, and if provider physician status is positive, said variable value is 1.

In an embodiment, for the one or more continuous variables, outliers are removed using a kernel density estimation approach. In an embodiment, for the one or more continuous variables, outliers are removed by removing variables having a probability density lower than a predetermined percentage of a maximum density. In an embodiment, the plurality of trials includes all permutations for the one or more models and the one or more parameter configurations.

Provided may be a non-transitory computer readable medium having a set of instructions stored thereon that, when executed by a processing device, cause the processing device to carry out an operation of treatment adherence prediction, the operation comprising receiving a full dataset; isolating a training dataset and a test dataset from the full dataset; splitting the training dataset into one or more training folds and a validation fold; training, over a plurality of trials, one or more models on each of the one or more training folds for each of one or more parameter configurations correlated to said one or more models, each of the one or more models configured to classify a target variable, wherein the target variable is an indicator of whether a patient will complete a treatment; validating, for each of the one or more models for a given trial of the plurality of trials, a given model of the one or more models on the validation fold; comparing a training score with a validation score, wherein the training score is based on the training of the one or more models over the one or more training folds, and wherein the validation score is based on the validating of the given model on the validation fold; recording classifications scores for each of the one or more models, the classifications scores based on the training score and the validation score for each of the one or more models; selecting a selected model from the one or more models, the selected model having a top classification score; retraining the selected model on the full dataset; and generating adherence predictions for the test dataset.

Provided may be a system for treatment adherence prediction, the system comprising a server comprising at least one server processor, at least one server database, at least one server memory comprising a set of computer-executable server instructions which, when executed by the at least one server processor, cause the server to receive a full dataset; isolate a training dataset and a test dataset from the full dataset; split the training dataset into one or more training folds and a validation fold; train, over a plurality of trials, one or more models on each of the one or more training folds for each of one or more parameter configurations correlated to said one or more models, each of the one or more models configured to classify a target variable, wherein the target variable is an indicator of whether a patient will complete a treatment; validate, for each of the one or more models for a given trial of the plurality of trials, a given model of the one or more models on the validation fold; compare a training score with a validation score, wherein the training score is based on the training of the one or more models over the one or more training folds, and wherein the validation score is based on the validating of the given model on the validation fold; record classifications scores for each of the one or more models, the classifications scores based on the training score and the validation score for each of the one or more models; select a selected model from the one or more models, the selected model having a top classification score; retrain the selected model on the full dataset; and generate adherence predictions for the test dataset.

In an aspect of the present disclosure, the system may utilize an algorithm. The algorithm may be configured to receive patient data (for example, demographic information or other identifying data derived from patient intake). The algorithm may then transform raw values to rescaled values (for example, “normalizing” the raw data). In such an embodiment, after the raw values have been rescaled, model weights may be applied to each of the scaled values. Thus, once the model weights are applied to the scaled values, a probability prediction may be generated.

In an embodiment, the probability prediction may be utilized to determine the likelihood that a particular patient will complete a predetermined portion of a treatment or drug regimen. Accordingly, using data collected as part of a standard intake evaluation, the algorithm may provide clinicians with rapid and accurate predictions to forecast the likelihood that a patient will complete said portion of treatment.

These and other aspects, features, and advantages of the present invention will become more readily apparent from the following drawings and the detailed description of the preferred embodiments.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 is an illustrative block diagram of a system based on a computer configured to execute one or more elements of the systems and methods described herein.

FIG. 2 is an illustration of a computing machine configured to execute one or more elements of the systems and methods described herein.

FIG. 3A is an illustration of an embodiment of algorithm development.

FIG. 3B is a workflow depicting an embodiment of the process of algorithm development.

FIG. 4 is a workflow depicting an embodiment of the algorithm.

FIG. 5 is a depiction of experimental results and performance of an embodiment of the algorithm.

FIG. 6 is a workflow depicting an embodiment of the process of algorithm development.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

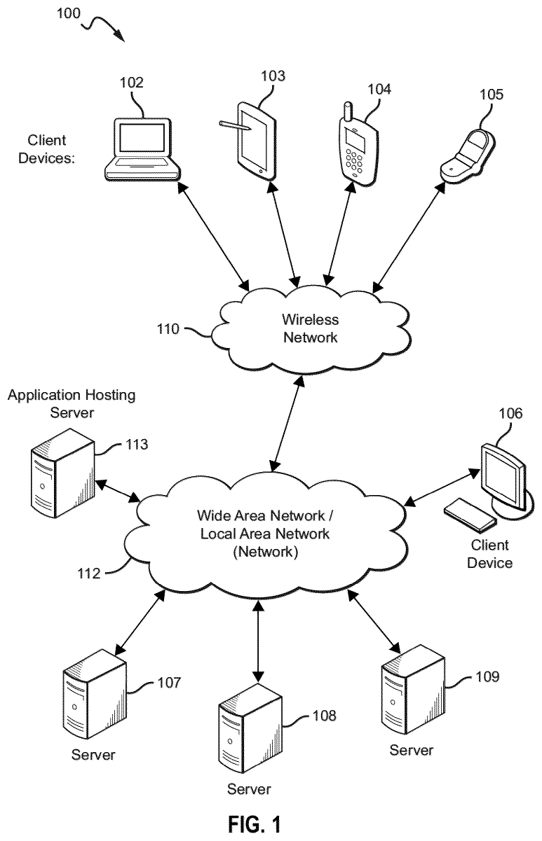

FIG. 1 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like. FIG. 1 also illustrates application hosting server 113.

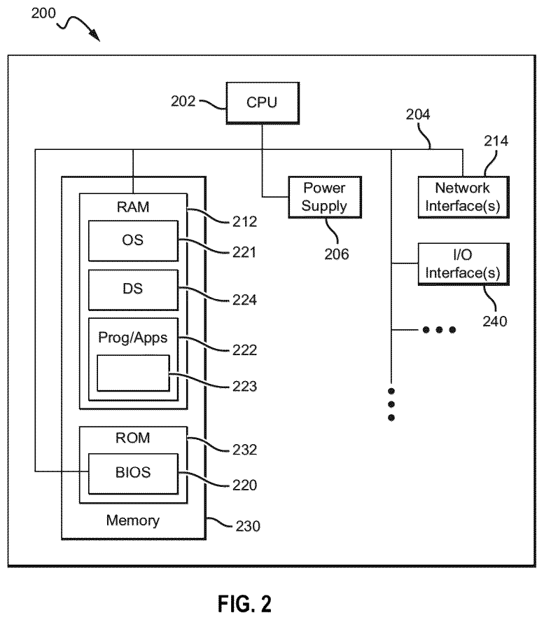

FIG. 2 illustrates a block diagram of an electronic device 200 that can implement one or more aspects of an apparatus, system and method for increasing mobile application user engagement (the “Engine”) according to one embodiment of the invention. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

The system described herein may utilize said component/devices 240 (e.g., biometric sensors) to capture relevant information for the EHRs and/or predictive algorithm described below. Accordingly, the aforementioned data sources (e.g., biometric sensors) may be considered one or more external sources. The biometric sensors (also referred to as digital biomarker-capturing devices) may be configured to capture one or more digital biomarkers from the patient. In an embodiment, the biometric sensors may be separate from the client device. In another embodiment, the biometric sensors may be integrated within the client device. Accordingly, information collected by the biometric sensors may be imported to the EHR described below and/or the treatment adherence algorithm described below. Thus, in an embodiment, the treatment adherence prediction may be based, at least partially, on the data collected by biometric sensors. Yet further, data retrieved from any smart phone, wearable, or other device and/or components thereof (e.g., microphones, touchscreens, keyboards, mice, GPS components, cameras, light sensors, accelerometers, etc.) may be implemented in the predictive algorithm described below. In an embodiment, the utilization of biometric sensors and other peripherals may allow the system to readily update the predicted treatment adherence with data retrieved “on the fly.”

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components, and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can ease processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MIMS, playing games (such as fantasy sports leagues), receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods implementing the Engine. Embodiments of the present invention may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

As noted above, embodiments of the present invention may relate to apparatuses, methods, and systems for forecasting treatment adherence based on data retrieved during patient intake. The embodiments may be referred to as the likelihood of treatment adherence system or simply, the “System.”

The System may utilize the computerized elements as described above and as illustrated in FIGS. 1-2. Accordingly, the System may include hardware and software elements configured to execute the features of the algorithm described herein.

The algorithm described herein may be developed in view of a large sample of patients and may be improved and/or accurized via suitable statistical methods. In effect, the System and the algorithm thereof may employ evidence-based medicine as informed by large samples of patients and valid statistical analysis. In an embodiment, the algorithm weighs all the inputs for each patient to arrive at a single personalized outcome prediction. In such an embodiment, the System, via the algorithm, may calculate and/or distribute an easily interpreted probability estimate. Such an estimate may be used to create a software feature in any suitable electronic health record (EHR) platform. However, the System may calculate and/or deliver the estimate absent an EHR platform. The estimate (also referred to herein as the probability estimate, probability, treatment adherence probability, and the like) determined herein may enable clinicians to make a data-driven decision on whether to offer a treatment (for example, including administration of ketamine) to a particular patient. The System described herein provides improvements to the technology of treatment adherence prediction systems by increasing the breadth of input data available for training the model(s), wherein said input data covers a diverse set of personal and clinical background information. Further, the System described herein improves upon the technology of treatment adherence prediction systems by increasing the predictive precision by training the model(s) on the large geographically diverse samples available through the platform described herein. Accordingly, in an embodiment, the treatment adherence probability is calculated with algorithms (e.g., classifier models) that have previously been trained on highly-relevant data with parameter settings that have been tailored to manifest accurate results.

In an embodiment, the System is configured to generate predictions based on standard intake data. For example, multiple data points may be collected during a standard intake examination. Such data points may be combined and converted into a single probability estimate. Predictions may be generated based on standard intake data and/or additional types of data. For example, such additional types of data include, but are not limited to, intake data, clinical notes, patient reported outcomes, demographics, psychiatric history, medical history, social history, family history, medication history, diagnoses, and allergies. In various embodiments, one or more of the aforementioned additional types of data are included in a standard intake, while some others are generated during a clinical encounter rather than the intake process. As a non-limiting example, during a routine visit the clinician could ask the patient about their family history and input that data, rather than having that information already input during the intake.

In an embodiment, the System described herein may utilize the categories of input data described in the feature list below. However, in further embodiments, the System may be configured to receive and analyze additional input data points. As a non-limiting example, such additional input data points may be provided electronically or verbally (for example, via a microphone embedded within the user's device) by the patient and may be entered into the electronic health record corresponding to said patient. As another non-limiting example, additional input data points may be measured from passive-sensing (e.g., a patient may connect their smart device or wearable to the System, wherein the smart device or wearable may upload activity history and utilize such data as an additional input). Moreover, as described in further detail below, the System is operable if individual data fields are missing. The System described herein may be adapted to be agnostic to all data intakes and/or may be configured for use with a universal data intake. The System may include a dedicated intake module configured to perform intake within the platform used to collect some of the input data. In an embodiment, the probability estimate ranges from 0 to 1 (non-inclusive), however, the probability estimates may be represented in percentages, fractions, ratios, or other suitable forms. The probability estimate may indicate the likelihood that an individual patient will complete a predetermined treatment threshold. The predetermined treatment threshold may be determined experimentally and/or may be set by a System administrator. However, the predetermined treatment threshold may be modified and/or otherwise altered during System operation and/or between instances of use. Further, the predetermined treatment threshold may be specific to a particular type of treatment and/or a particular patient or class of patients. As a non-limiting example, the predetermined treatment threshold may include at least 4 infusions of IV ketamine within 28 days of the intake examination. However, the predetermined treatment threshold may be any measure of the completeness of a particular treatment regimen. Thus, as an example, the predetermined treatment threshold may be represented as a sufficient dosage or duration of treatment relative to the preferred dosage or duration of treatment. In an alternate embodiment, the predetermined treatment threshold may be a range of values (e.g., 4-8 infusions of IV ketamine within 28 days).

The algorithm and the host System may utilize data retrieved from clinical trials, studies, patient intake, and/or other sources of experimental data. As non-limiting examples, sources of data available to the System include, but are not limited to, data retrieved via a patient-mobile application, data stored within a provider electronic health record application, data captured via a patient electronic intake survey, or data from an electronic-prescribing platform. In an embodiment, the System may retrieve third-party data from pharmacies and other electronic health records available through health informatics exchanges. Accordingly, such expanded datasets may provide for improved algorithm development.

In one embodiment, the data may be retrieved from various locations, for example, to develop an algorithm based on both a diversity of locale and diversity of patient. The sources of the data may either have dispensed or observed administration of the target treatment and/or drug and thus correlate to the patients thereof. For example, the dataset may include a number of individuals who have completed an initial consultation for IV ketamine treatment for depression. Therefore, the dataset may include both the initially received data (e.g., standard intake data) and compliance/adherence data relative to the target treatment and/or drug.

The data utilized by the algorithm may be retrieved from clinical measures or processes known to persons of ordinary skill in the art. However, the data may be retrieved from any suitable measures or processes. As a non-limiting example, such data retrieval may include, but is not limited to, Quick Inventory of Depressive Symptomatology (QIDS-SR16, hereafter referred to as “QIDS”); Generalized Anxiety Disorder-7 (“GAD-7”); Psychiatric evaluation for ICD-10 diagnoses of mood and anxiety disorders; and/or demographic assessment of age, race, ethnicity, and/or sex. Accordingly, as to decrease required deviation from standard clinical procedures, each measure may be a standard assessment that a clinician may perform when conducting an intake evaluation for treatment of the target condition (e.g., depression). Therefore, the data points generated by these measures and used by the algorithm may include, but are not limited to, the QIDS total score; the GAD-7 total score; for demographics, 4 variables with the default value of 0 (for example, values are 1 if patient is: 1. male sex, 2. missing sex data, 3. Caucasian, 4. not Caucasian); age; for ICD-10 diagnoses, 4 variables with the default value of 0 (for example, values are 1 if patient has a diagnosis of: 1. major depression, 2. an anxiety disorder other than post-traumatic stress disorder [PTSD], 3. PTSD, 4. bipolar disorder); and a count of the number of different ICD-10 diagnoses a patient has. However, the aforementioned measures should not be viewed as limiting, the metrics analyzed by the algorithm may include any number or combination of variables set to any suitable default values or thresholds.

In further embodiments, data retrieved from intake may include the patient's history of motion sickness and/or vertigo (for example, as a predictor of noxious side-effects during KIT). In an embodiment, historical variables may include those computed from the provider entity/clinic-side data, such as indicators of the clinic's historical experience with delivery of intravenous ketamine treatments or other relevant treatments. Accordingly, one or more data points may be correlated to the clinic(s) offering the relevant treatment. Thus, the algorithm, and resulting predicted treatment adherence, may be a function of the patient's clinic's history and other characteristics. As a non-limiting example, if a particular patient is enrolling in a particular clinic that has shown substantial treatment adherence, the algorithm may provide a greater probability to said patient's success versus a patient enrolled in a subpar clinic. However, the data retrieved from intake may include any data points, variables, and/or may illuminate any such connections between intake data and the underlying treatment.

As described above, each treatment may include a corresponding target variable (e.g., a predetermined treatment threshold). For the purposes of IV ketamine infusions, the desired target variable for the corresponding dataset may be an indicator of whether a patient completed the prescribed minimum initial course (induction) of IV ketamine infusions. In such an embodiment, the minimum initial course is defined as completing at least 4 infusions within 28 days from the intake evaluation. However, the target variable (predetermined treatment threshold) may be any measure of completeness, such as full course completion, completion of an initial course, or completion of a desired percentage or stage of the treatment regimen. Thus, for the purpose of training statistical models to predict said target variable, patients who completed the minimum course may be assigned a value of 1 and the remaining patients may be assigned a value of 0. However, the patients may be assigned any value between 0 and 1 correlating to their associated treatment completion status, or any numerical value representing a measure of the desired level of treatment adherence. As a non-limiting example, after an initial intensive 4-week long “induction” phase, some patients continue receiving maintenance infusions for an extended period of time. Accordingly, in such a non-limiting example, a tailored algorithm could be developed to predict this type of long-term continuation of treatment. Additional target variables for development may be defined by the degree of patient clinical response to said treatment and/or the clinical response or adherence to other treatments. Thus, in an embodiment where the System contemplates multiple target variables, the System could increase clinical value by predicting differently-defined target variables.

As a further non-limiting example, alternative models may be configured to predict a number outside the range of 0 to 1. Moreover, any suitable statistical analysis metrics and methods may be utilized. As described above, any target variable and appropriate completion metric may be established for a particular treatment. The System described herein may be utilized for predicting the adherence to any treatment, wherein said treatment may have a completion metric defined, for example, by the System administrator. In an embodiment, the target variable may be an induction course, an initial schedule of medication, the complete treatment regimen, or any other suitable duration or treatment goal. The ability to define the target variable may allow the algorithm to be tailored to any treatment.

The System may utilize one or more statistical models used to separate units into “classes,” such as “treatment dropout” and “treatment completer,” which may be referred to as classifiers (also referred to herein as “classifier model”). In effect, a classifier model may be selected based on the data stream such that the classifier most accurately predicts the target. In an embodiment, experiments are executed to produce a classifier to exhibit increased accuracy when generating predictions for future patients with data that was not used to train the model.

In an embodiment, a “test set” may be created by removing a predetermined percentage (for example, 30%) of the sample and saving the test set for validation after training of multiple candidate models for selection. Thus, the “training set” may comprise the remaining percentage (e.g., 70%) of data. A sequence of model training trials may be executed utilizing the training set. Each trial may test a unique combination of parameters including, but not limited to the type of classifier (for example, Lasso Logistic Regression and/or Ridge Logistic Regression); the value of the regularization strength of the classifier (for example, controlling how strongly the model fits to individual data points); and the probability threshold for predicting a patient as a “completer” (for example, with 4 values tested). Accordingly, the parameters (including those parameters known as hyperparameters) may be modified or tuned for each trial. Thus, the parameters may be dependent on the type of model being implemented. For example, for a Logistic Regression model, the solver and/or the regularization may be tune. As another example, for a K-Nearest Neighbors model, the number of neighbors, the distance metric, and/or the weight contributions from neighbors may be tuned. However, each type of model may have the corresponding parameters tuned and none of the examples described herein should be interpreted as limiting.

For each of the trials, the classifier may be “fit” to the training data. For example, the classifier may attempt to learn the mathematical relationship between the inputs and the target variable. In each trial, the unique combination of parameters (described above) may impact how the model fits to the training data. Consequently, the fitted models from each of the trials may differ from one another. In an embodiment, multiple trials may be repeated utilizing different parameters as to discover the combination of parameters that produce the most accurate predictions for “out-of-sample” data (for example, data that the model did not use for fitting).

In an embodiment, to assess outcomes in adherent versus non-adherent patients, the non-limiting procedure below may be utilized. First, in such a non-limiting example, gather all questionnaires (or other data) from a predetermined period of time (e.g., 30 days) prior to the first treatment date to a predetermined period of time (e.g., 180 days) after the first treatment. Next, in such a non-limiting example, within patients, take the average of all questionnaires (or other data) in the following time bins: (i.) baseline: [−30, 0] from first treatment; (ii.) weekly from (0, 28] days from first treatment (for example, for visualization only, these bins may not be included in the models); and (iii.) monthly from (28, 180) days from first treatment (for example, the first month may include days 29-60, all other months may include 30 days). In a further embodiment, for each questionnaire (or other data), utilize a linear mixed effects model with the following predictors: (i.) intercept (the baseline outcome) with separate intercepts for each patient; (ii.) time bins where each time bin is treated independently, such that the model estimates a coefficient representing the average difference from baseline to each time bin, manifesting a separate effect of time bin for each patient; (iii.) “time bin x adherence interaction,” representing the difference between adherent and non-adherent groups at each timepoint (for example, including the baseline timepoint); and (iv.) the number of maintenance infusions during that month.

In an embodiment, including random (i.e., individual) effects of the intercept (baseline outcome) and effect of time bins may be important to account for error in model predictions due to repeated measures (e.g., that patients with a higher baseline may be more likely to have higher scores at later timepoints or that patients with higher baseline scores are more likely to have a greater reduction due to treatment).

In an embodiment, to test for differences in the number of maintenance treatment components (for example, scheduled medications) on a month-by-month basis (the same time bins in which outcomes may be analyzed), the non-limiting procedure below may be used. First, in such a non-limiting procedure, calculate the empirical cumulative distribution (ECDF) function at each time point for adherent and non-adherent patients. Next, in such a non-limiting procedure, test for differences at any point along the ECDF using a Kolmogorov-Smirnov (KS) test. Further, in such a non-limiting procedure, adjust p-values for multiple comparisons using Bonferroni correction. Such a procedure may determine that a greater proportion of adherent patients receive more treatment components than non-adherent patients at all time points tested. In such an instance, although the clinical difference may be small, the sample size may be large enough to determine that there is a statistical difference between groups.

Provided below is a non-limiting example of a model selection procedure. In such a non-limiting procedure, using the same set of features, 4 different machine learning techniques may be evaluated: (i.) (Regularized) logistic regression; (ii.) Bayesian logistic regression; (iii.) Random forest classification; and (iv.) Naive bayes classifier. Models may be compared via their cross-validated log likelihood score, calculated only using the “Training Set,” wherein the “Training Set” may be split into 5 folds, wherein each model may be estimated on 4 of the folds and evaluated on the 5^{th}, and wherein this procedure may be repeated such that each fold is held out once. In a further embodiment, the logistic regression and random forest models have hyperparameters that define the complexity of the model. In an embodiment, for these models, nested cross-validation was used, wherein cross-validation may be performed on the 4 training folds to determine the optimal hyperparameters for that data, wherein the model may be fit to the 4 training folds using the optimal hyperparameters, and wherein this model may be used to generate predictions for the left out fold. As a non-limiting example, hyperparameter values tested for Logistic regression may include, (i.) penalty value: [0.001, 0.01, 0.1, 1, 2, 4, 8, 16, 32, 64, 128, 256, 512], and (ii.) 11 ratio (for elasticnet penalty): [0, 0.1, . . . , 0.9, 1]. As a non-limiting example, hyperparameter values tested for Random forest may include, (i.) number of trees: [2, 4, 8, 16, 32, 64, 128, 256, 512, 1024], (ii.) max tree depth: [1, 2, . . . , 6, 7], and (iii.) criterion function: [Gini impurity, Shannon information gain].

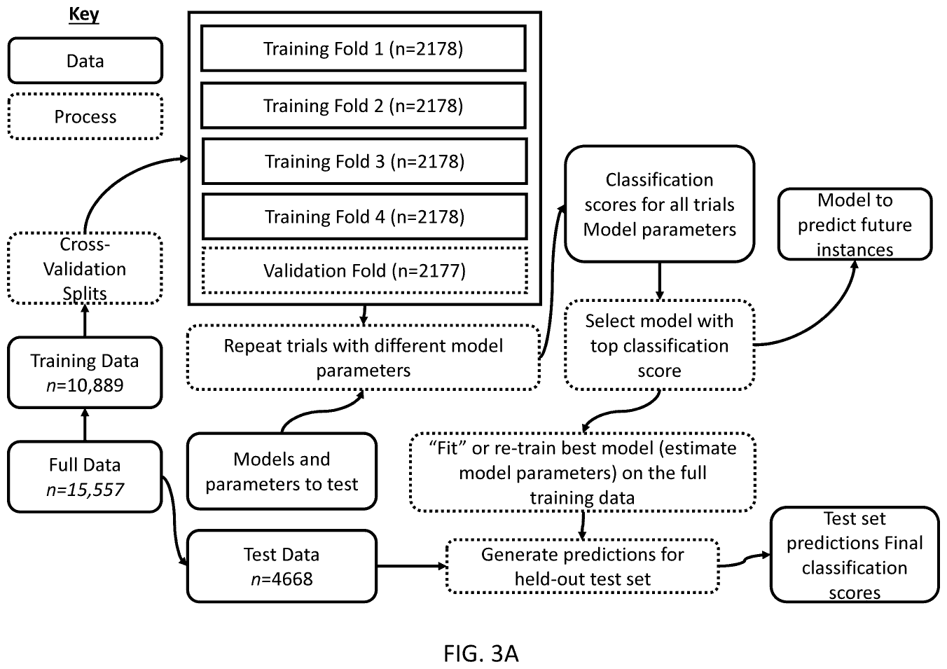

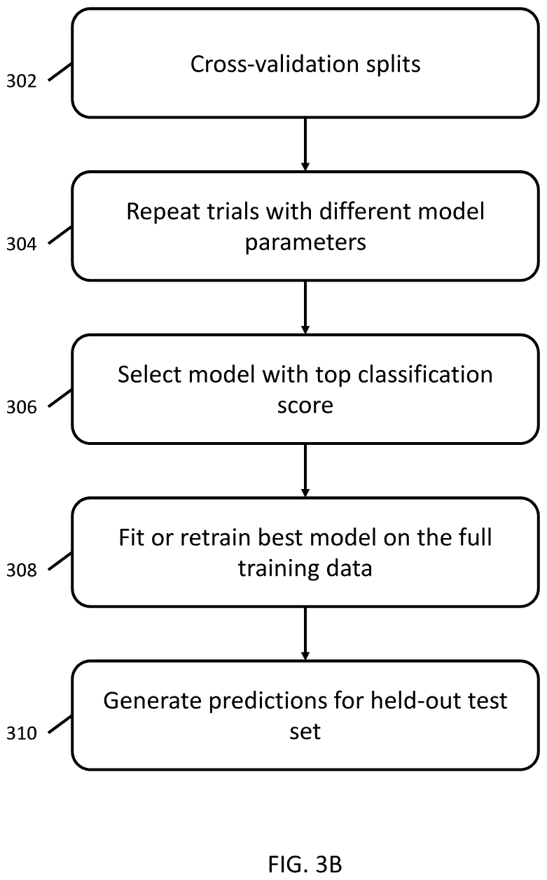

As shown in FIG. 3A, from all of the models that are tested, the best model may be selected according to a set of scores collected for each trial. In an embodiment, scores may be generated using a process of “cross-validation.” For example, for each trial, the model may be configured to fit data comprising most of the training set (for example, an 70% subset of the training set), and the fitted model may be utilized to predict the validation fold (for example, the remaining 30% of the training set). In each trial, this process may repeat for a number of iterations (for example, five, corresponding to one iteration each for prediction of each 30% validation fold). The process may repeat once for each sector of training data, with predictions made for the remaining 30%. The resulting predictions may be stored for each trial and each trial iteration. In an embodiment, these predictions are then compared to the true values in order to compute a set of scores for each trial. Following the cross-validation process, a model may be fit to the entirety of the training set and may be utilized to generate a probability prediction of the test set. Such a procedure may produce estimates of future “out-of-sample” performance, providing insight to a reproducible selection of parameters for implementation. FIG. 3A displays a non-limiting exemplary sample size of n=2,350, however, any suitable sample size may be used and the test data, training data, training folds, and/or validation folds may be any suitable subsets thereof.

In sum, as shown in FIG. 3A, algorithm development may include the processes of splitting initial data into test data and training data, wherein the training data is utilized in a cross-validation process. For example, the training data may be further split into one or more training folds and one or more validation folds. In one embodiment, the model may be fitted on data from four folds and the fitted model may predict the fifth fold. Such a process may be repeated a selected number of times (for example, four times) to get predictions for each fold. Therefore, the “cross-validated” predictions may be collected, and predictions may be compared to their true target values.

The trials may be repeated with different model parameters and models to determine those that are most accurate and/or deliver a model best-equipped to predict treatment adherence likelihood. Classification scores may be determined for all trials and model parameters. Therefore, the model with the highest classification score may be selected and such a model may be utilized in future instances. In such an embodiment, said model may be utilized to generate predictions for the held-out test set data (for example, previously split from the initial full data set). Accordingly, the test set predictions may be evaluated for final classification scores.

In sum, referring to FIG. 3B, the process of algorithm development and execution may being with, step 302, cross-validation via splitting the training data into training folds and validation fold(s). At step 304, the trials may be repeated for each permutation of different model and parameter. Accordingly, at step 304, each trial may represent an attempt at producing the most accurate results with a model by utilizing a uniquely tuned model parameter schema. At step 306, the model that has performed with the highest classification score may be selected. At step 308, the selected model may be retrained on the full training data, allowing the selected model to be further fine-tuned. At step 310, predictions may be generated with the selected model for the held-out test set. Ultimately, the model defined by the aforementioned process may be tuned to classify treatment adherence for a particular treatment. In various embodiments, the aforementioned process may be utilized to train and tune models for any treatment.

In an embodiment, for each type of classifier, there may be a list of settings that can be altered. Said list may depend on the structure of the classifier. Accordingly, a system developer may select a list of values to try for each setting. For example, such a setting test could be devised as a finite list of specific values (“grid search”) or it could be devised as a range within which to test a specified amount of randomly selected values (“random search”). In such an example, once the list of values to try for each setting is determined, each “trial” may fit the model to the data using a unique combination of values across all the settings for that classifier type. In the non-limiting example where a classifier has two specified values for both of two settings, four total trials would be run, one for each unique combination of values. Accordingly, after running a series of trials and inspecting the results, another series of trials may be run using a different set of values to maximize classifier performance on the cross-validated predictions. In such an example, each iteration seeks to identify the combination of settings from early runs with the best performance, eliminate values with worse performance, and improve the performance on cross-validated predictions on subsequent runs.

As an example, the mathematical relationship between the inputs and the estimated probability of IV ketamine adherence may be represented, in part, by a probability function p(x), where p (x) is defined as the probability that a given patient will complete at least 4 sessions of IV ketamine treatment. However, the p(x) function may be altered and/or otherwise modified to determine the probability of completion of any portion or milestone of any treatment and/or drug regimen. Thus, the p(x) function should not be viewed as limited to the example of IV ketamine treatment. The algorithm may utilize:

$$p ( x ) = \frac{1}{1 + {e}^{- ( f )}}$$

wherein f is a linear formula defined by one constant intercept and 37 multiplicative coefficients applied to 37 measured variables as such:

f=β₀+β₁x₁+β₂x₂+β₃x₃ . . . +β₃₇x₃₇

wherein β₀ is a constant value and β_{1 . . . 13} are coefficients.

The 37 variables in f may be defined as such:

| | |
|---|---|
| x₁ | Normalized population density of patient's resident zip code |
| x₂ | Normalized median income of patient's resident zip code |
| x₃ | Normalized median home of patient's resident zip code |
| x₄ | Normalized number of total ICD-10 diagnoses |
| x₅ | Normalized number of ICD-10 diagnoses considered as psychiatric conditions |
| x₆ | Normalized number of patients clinic has previously treated with KIT |
| x₇ | Normalized proportion of prior patients at the clinic that met the threshold for |
| | adherence to KIT |
| x₈ | Normalized patient's age at first infusion |
| x₉ | Normalized patient's BMI |
| x₁₀ | Normalized number of days patient had been associated with the clinic prior to |
| | their first KIT treatment |
| x₁₁ | Normalized GAD7 Composite Score |
| x₁₂ | Normalized PHQ9 Composite Score |
| x₁₃ | GAD-7 Item 1 Score |
| x₁₄ | GAD-7 Item 2 Score |
| x₁₅ | GAD-7 Item 3 Score |
| x₁₆ | GAD-7 Item 4 Score |
| x₁₇ | GAD-7 Item 5 Score |
| x₁₈ | GAD-7 Item 6 Score |
| x₁₉ | GAD-7 Item 7 Score |
| x₂₀ | PHQ-9 Item 1 Score |
| x₂₁ | PHQ-9 Item 2 Score |
| x₂₂ | PHQ-9 Item 3 Score |
| x₂₃ | PHQ-9 Item 4 Score |
| x₂₄ | PHQ-9 Item 5 Score |
| x₂₅ | PHQ-9 Item 6 Score |
| x₂₆ | PHQ-9 Item 7 Score |
| x₂₇ | PHQ-9 Item 8 Score |
| x₂₈ | PHQ-9 Item 9 Score |
| x₂₉ | 1 if patient is male, 0 otherwise |
| x₃₀ | 1 if patient is in relationship, 0 otherwise |
| x₃₁ | 1 if patient filled out an intake form for the clinic, 0 otherwise |
| x₃₂ | 1 if patient is diagnosed with a mood disorder, 0 otherwise |
| x₃₃ | 1 if patient is diagnosed with an anxiety disorder, 0 otherwise |
| x₃₄ | 1 if patient is diagnosed with an attention disorder, 0 otherwise |
| x₃₅ | 1 if patient had a visit at the clinic prior to their first KIT treatment, 0 otherwise |
| x₃₆ | 1 if patient's provider is a physician, 0 otherwise |
| x₃₇ | 1 if patient's provider has an advanced nurse or nurse practitioner credential 0 |
| | otherwise |

However, the 37 variables in f may be defined as any suitable measures or metrics derived from patient intake data. Additionally, f may include any suitable number and/or combination of variables.

As a non-limiting example, the normalization formula for numeric variables may be defined as:

xᵢ=(x_{raw}−xₘᵢₙ)÷(xₘₐₓ−xₘᵢₙ)

wherein terms may be defined as:
xᵢ=Value of x input to algorithm
x_{raw}=Original measured value of x
xₘₐₓ=Sample maximum value of x
xₘᵢₙ=Sample minimum value of x.

In an embodiment, the normalization approach is dependent on whether the variable is a continuous variable or a binary variable. In an embodiment, for continuous variables (i.e., variables whose values are integers or real numbers and not a simple true or false), outliers values may be removed, for example, using a kernel density estimation approach. Accordingly, the probability density function of the variable may be estimated, any variables with a probability density lower than a predetermined percentage of the maximum density may be removed. A power transformation may be applied to reshape the distribution of values to be approximately Gaussian (xₜ=transform(x)). In an embodiment, missing variables may be ignored during the reshaping of distributions. The values may be scaled using a z-transformation, for example: x_{z}=(xₜ−mean(xₜ))/st_dev(xₜ). Missing values may be ignored during the z-transformation. Missing values may be “imputed,” wherein a value of 0 may be inserted for all unknown values or values that were removed as outliers earlier. In an embodiment, for binary variables, variables may be coded as 1 if true, −1 if false, and/or 0 if missing. Although the above means of normalization may be utilized for one or more of the models described herein, some classifiers may utilize additional normalization steps, which are contemplated below.

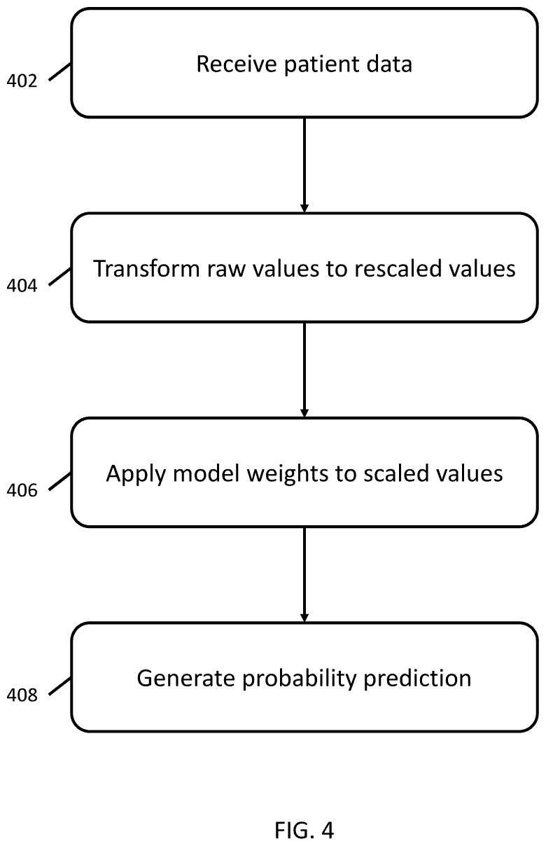

In sum, as shown in FIG. 4, at step 402, the algorithm may first receive patient data (for example, demographic information or other data derived from patient intake). The algorithm may then, at step 404, transform raw values to rescaled values (for example, as described herein as “scaling” and/or “normalizing”). After the raw values have been rescaled, at step 406, model weights, as described herein, may be applied to each of the scaled values. Thus, once the model weights are applied to the scaled values, at step 408, a probability prediction may be generated via the probability function. The steps of normalizing, weighting scaled values, and calculating a probability prediction via the probability function may be implemented via the computerized components and networks as disclosed above and in FIGS. 1-2. The architecture of the algorithm may not reside on a single server, but rather a container that may be deployed on-demand across one of many servers (i.e., AWS servers). However, in an alternate embodiment, the architecture of the algorithm may reside on a single server.

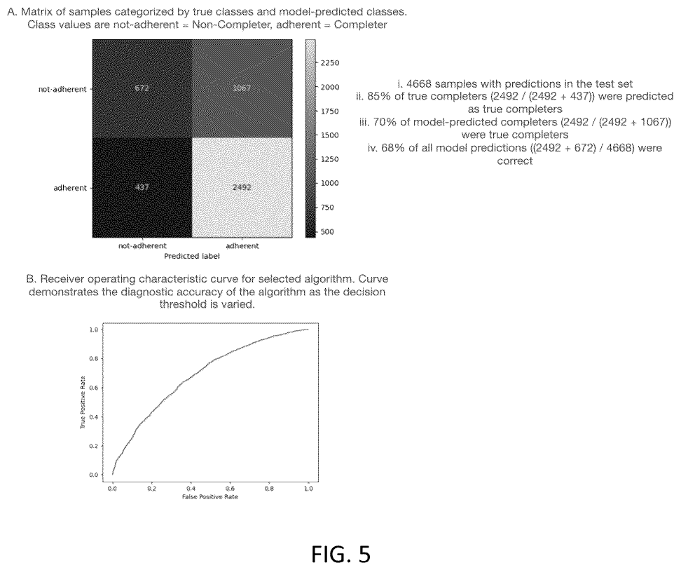

FIG. 5 displays metrics of classifier accuracy generated from out-of-sample predictions of the selected algorithm. These metrics and similar metrics may be a source of evidence that the selected algorithm accomplished the desired technical solution.

In alternate embodiments, different data points may be used as inputs to the algorithm, (either by removing a number of inputs, adding new inputs, or replacing a number of inputs with others). As a non-limiting example, the PHQ-9 depression measurement may be replaced and/or supplemented with a different measurement metric, such as QIDS. Accordingly, modification of the inputs may alter the algorithm. The algorithm may assign different weights to existing inputs if other inputs were added or were removed, and any replacement inputs may also have a different weight assigned to them.

In another alternate embodiment, a different type of classifier may be implemented. The classifier may be a “linear” model, wherein the form of the algorithm is restricted to a linear formula. However, the classifier may be nonlinear in nature, such as decision tree models or K-Nearest Neighbors models. The classifier may also be characterized as an “ensemble” model, wherein the function of the classifier may be generated by a combination of multiple individual classifiers. Such an ensemble model may not be sufficiently described in a single formula as displayed above. “Ensemble” may refer to any type of model where multiple individual models (aka “base estimators”) contribute to the predictions. As a non-limiting example, two distinct types of models (e.g., logistic regression and naive Bayes) could be developed using the same features dataset and target. In one such instance, the first model type predicts a positive class probability of 0.80, the second model type predicts a positive class probability of 0.60, and the ensemble algorithm may average both probabilities to produce a final probability of 0.70. As a further non-limiting example, the System may be equipped to utilize algorithms commonly implemented as ensembles in standard machine learning software packages (e.g., a random forest model, which is an ensemble of decision trees, where the predictions of tens, hundreds, or even thousands of decision trees can be averaged to arrive at the final prediction).

The classifier may also be characterized as a “black box” model, wherein the transformations applied to the data inputs may occur in a process comprising multiple individual steps that are dynamic and interdependent. Similarly, such a black box model may not be sufficiently described in a single formula as displayed above. Further, such a black box model may be applied through application of a trained model directly in software. In an alternate embodiment, the System may include “stacked” implementations of algorithms, wherein the output(s) of one algorithm may be fed as inputs to one or more additional algorithms to produce the final predictions. Additional classifiers may include: Bayesian linear models (which may be similar to linear models, but estimate the uncertainty in the relationships between model variables and outcomes); hierarchical models (which model parameters may have different values based on their relationships with other variables. For example, the “weight” for PHQ-9 scores may be different for patients who are treated by a physician vs. patients who are treated by a provider with an advanced nursing credential); naive Bayes classifiers (which leverage Bayes Theorem to estimate how likely a set of variables is to appear in one “class” vs. another); and/or kernel-based methods, including but not limited to Support Vector Machines, Relevance Vector Machines, and Gaussian Process Classification, which use linear methods on data that is transformed to a high-dimensional, non-linear space.

In further embodiments, to improve the algorithm performance, additional data points may be included to provide new inputs to the model. There may be additional explanatory variables that, when included, aid in error correction and/or improve overall classification performance. The present disclosure contemplates the possibility that derivations of existing input data may improve performance when added to the feature set. For example, additional new variables computed from diagnostic data or multiplicative combinations of features may add additional explanatory power to predictive models. Additionally, data integrated with smartphones and/or biotech assessments (e.g., brain electrical activity measures) may be utilized.

Such additional inputs may be derivatives of the included inputs (for example, the product of two or more inputs or the squared value of a single input). Alternatively, such additional inputs may be derived from new measures and data points not included in the initial inputs. In one embodiment, inputs may be removed to improve performance of the algorithm.

Further, as described above, any suitable classifier may be implemented.

In another embodiment, the initial sample size of data may be increased as to better fit the algorithm. For example, an increase in the sample size of the data may be utilized to accurize the classifiers for training.

Further, the System may be configured for updating the model's predictions in “real-time” as a patient progresses through the course of treatment. For example, an algorithm trained on the set of patients who have completed a particular milestone (for example, at least 1 ketamine infusion) may be utilized to illuminate evaluation at that point in the course of treatment (for example, as compared to the predictions served after the patient's initial evaluation).

The algorithm as described herein may be modified by alteration of the target variable. As a non-limiting example, instead of training the model on treatment adherence, the algorithm may be trained to predict whether a patient has remission from depression after IV ketamine treatment.

The algorithm described herein may be configured to make accurate predictions on out-of-sample data. Accordingly, the algorithm may be adapted to utilize a machine learning framework in conjunction with readily acquired standard patient intake data to predict likelihood of adherence to prescribed IV ketamine treatment for a particular patient.

The algorithm may include transforming the raw values of clinical measures (for example, QIDS, GAD-7) into rescaled values. This step, often referred to as “Scaling” or “Normalizing” the data, may be taken both when fitting the model to training data in the data experimentation workflow outlined in FIG. 3A and when the model is implemented in software for making predictions on new samples. This step may improve both the data experimentation workflow and the software implementation. Accordingly, normalizing the data increases accuracy of predictions because, for some inputs, the algorithm's weights may be either much too small or too large for the unscaled data.

The algorithm described herein may be executed and/or used in connection with any suitable machine learning, artificial intelligence, and/or neural network methods. For example, the machine learning models may be one or more classifiers and/or neural networks. However, any type of models may be utilized, including regression models, reinforcement learning models, vector machines, clustering models, decision trees, random forest models, Bayesian models, and/or Gaussian mixture models. In addition to machine learning models, any suitable statistical models and/or rule-based models may be used.

In an embodiment, the desired target variable for a dataset may be an indicator of whether a patient completed the prescribed full initial course (induction) of the particular treatment (e.g., IV ketamine infusions). The full initial course may be defined as completing a predetermined portion of the treatment (e.g., at least 4 infusions within 28 days from the intake evaluation).

In an embodiment, patients who completed the full course may be assigned a 1 and all other patients may be assigned a 0, for the purpose of training statistical models to predict said target.

The algorithm and underlying classifier may be determined by seeking a classifier that predicts the target as accurately as possible. More specifically, methods (for example, as shown in FIG. 3A) may be executed to produce a classifier to be as accurate when making predictions for future patients with data that was not used to train the model.

Accordingly, a “test set” may be created by removing a predetermined portion of the sample and holding it out for later testing. The remaining portion of data (the “training set”) may be utilized for a sequence of model training trials. Each trial may test a unique combination of each of the parameters. Parameters may include the type of classifier, the value of regularization strength of the classifier (for example, with a predetermined number of options tested), the probability threshold for predicting a patient as a “completer” (for example, a predetermined number of values tested).

For each of the trials, the classifier may be “fit” to the training data. For example, in “fitting” the classifier attempts to learn the mathematical relationship between the inputs and the target variable. In an embodiment, for each trial, the unique combination of parameters may impact how the model fits to the training data. In such an embodiment, as a result, the fitted models from all the trials may differ from each other, even when all are using the same data for model fitting. The purpose of repeating multiple trials with different parameters may be to discover the combination of parameters that produce the most accurate predictions for “out-of-sample” data (data that the model did not use for fitting).

From the models that were tested, the best model may be selected according to a set of scores collected for each trial. In an embodiment, scores are generated using a process of “cross-validation”. For each trial, the model may fit to a subset of the training data, and the fitted model may be used to predict the remaining portion. On each trial, this process repeats a predetermined number of times (once for each unique portion of training data, with predictions made for the remaining portion), and all predictions may be stored. These predictions may then be compared to the true values to compute a set of scores for each trial.

In an embodiment, the data inputs for the algorithm may be entered into a patient mobile or web application and/or a provider web application. All inputs may be stored in an electronic health record database. In an embodiment, a separate software “container” holds all the necessary programming to convert the raw data stored in the database to the patient's estimated probability of treatment completion. In an embodiment, this conversion follows a sequence of the following steps: each raw data point is converted into a rescaled value; according to the formula provided by the trained algorithm, a multiplier or “weight” is assigned to each input data point to calculate the change in probability associated with that specific input; and all inputs are summed to generate the final probability prediction.

In an embodiment, the algorithm software container stores the probability value in the electronic health record database and then the probability value may be made visible in the patient's chart in the web application of the electronic health record platform.

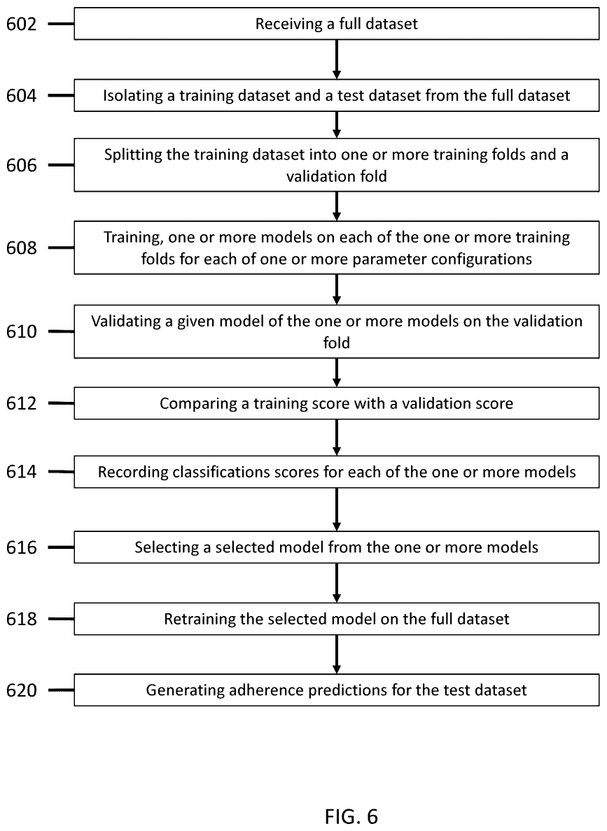

FIG. 6 is a workflow depicting an embodiment of the process of algorithm development. Referring to FIG. 6, the System may include a computer-implemented method. In an embodiment, said method may, at step 602, receive a full dataset, wherein the full dataset may be comprised of patient data. Further, the computer-implemented method may, at step 604, isolate at least a training dataset and a test dataset from the full dataset. Further, at step 606, the training dataset may be split into one or more training folds and/or a validation fold. Moreover, at step 608, the method may include training, over a plurality of trials, one or more models on at least the one or more training folds for each of one or more parameter configurations. In an embodiment, at step 610, the model may be validated on the validation fold. Said parameter configurations may be correlated to the one or more models. In an embodiment, the one or more models are configured to classify a target variable, wherein the target variable is an indicator of whether a patient will complete a treatment. The computer-implemented method may validate a given model of the one or more models for each of the said models. In a further embodiment, at step 612, the method may compare a training score against a validation score, wherein the training score is based on the training of the one or more models, and wherein the validation score is based on the validating of the given model. Additionally, the method may, at step 614, record classifications scores for each of the one or more models, wherein the classifications scores are based on at least one of the training score and the validation score. In an embodiment, at step 616, the method may select a selected model from the one or more models, wherein the selected model has a top classification score. In yet a further embodiment, at step 618, the method may retrain the selected model on the full dataset, wherein said retraining may, at step 620, generate an adherence prediction for the test dataset.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the claims.

1. A computer-implemented method for treatment adherence prediction, comprising the steps of:
receiving a full dataset;
isolating a training dataset and a test dataset from the full dataset;
splitting the training dataset into one or more training folds and a validation fold;
training, over a plurality of trials, one or more models on each of the one or more training folds for each of one or more parameter configurations correlated to said one or more models, each of the one or more models configured to classify a target variable,
wherein the target variable is an indicator of whether a patient will complete a treatment;
validating, for each of the one or more models for a given trial of the plurality of trials, a given model of the one or more models on the validation fold;
comparing a training score with a validation score,
wherein the training score is based on the training of the one or more models over the one or more training folds, and
wherein the validation score is based on the validating of the given model on the validation fold;
recording classifications scores for each of the one or more models, the classifications scores based on the training score and the validation score for each of the one or more models;
selecting a selected model from the one or more models, the selected model having a top classification score;
retraining the selected model on the full dataset; and
generating adherence predictions for the test dataset.

2. The computer-implemented method of claim 1, wherein the treatment comprises intravenous (IV) ketamine infusions.

3. The computer-implemented method of claim 2, wherein the completeness of the treatment is defined as the patient completing at least four IV ketamine infusions within twenty-eight days from an intake evaluation.

4. The computer-implemented method of claim 1, wherein the one or more parameter configurations are based on a list of settings associated with each of the one or more models.

5. The computer-implemented method of claim 1, wherein the one or more models are selected from a group of classifier types comprising Bayesian linear models, hierarchical models, naive Bayes classifiers, and kernel-based methods.

6. The computer-implemented method of claim 1, wherein at least one of the one or more model is an ensemble model.

7. The computer-implemented method of claim 1, wherein at least the training dataset comprises a plurality of variables, wherein each of the one or more models is configured to classify a target variable based on each of the plurality of variables.

8. The computer-implemented method of claim 7, wherein the plurality of variables comprises one or more continuous variables and one or more binary variables, wherein each of the one or more continuous variables is an integer or real number, and wherein each of the one or more binary variables is encoded as 1 if true, −1 if false, and 0 if missing.

9. The computer-implemented method of claim 7, wherein the plurality of variables comprises a normalized population density of a resident zip code, a normalized median income of a resident zip code, a normalized median home price of a resident zip code, a normalized number of total ICD-10 diagnoses, and a normalized number of ICD-10 diagnoses considered as psychiatric conditions.

10. The computer-implemented method of claim 9, wherein the plurality of variables comprises a normalized number of prior patients treated by a clinic with KIT, a normalized proportion of prior patients at the clinic that met a threshold for adherence to KIT, the patient's age at first infusion, the patient's BMI, and a normalized number of days patient had been associated with the clinic prior to their first KIT treatment.

11. The computer-implemented method of claim 10, wherein the plurality of variables comprises a normalized GAD7 composite score and a normalized PHQ9 composite score.

12. The computer-implemented method of claim 10, wherein the plurality of variables comprises a GAD-7 Item 1 Score, a GAD-7 Item 2 Score, a GAD-7 Item 3 Score, a GAD-7 Item 4 Score, a GAD-7 Item 5 Score, a GAD-7 Item 6 Score, a GAD-7 Item 7 Score, a PHQ-9 Item 1 Score, a PHQ-9 Item 2 Score, a PHQ-9 Item 3 Score, a PHQ-9 Item 4 Score, a PHQ-9 Item 5 Score, a PHQ-9 Item 6 Score, a PHQ-9 Item 7 Score, a PHQ-9 Item 8 Score, and a PHQ-9 Item 9 Score.

13. The computer-implemented method of claim 8, wherein the one or more continuous variables comprises sex, relationship status, completion status of intake form, mood disorder diagnosis, anxiety disorder diagnosis, attention disorder diagnosis, pre-visit status, and provider physician status.

14. The computer-implemented method of claim 13, wherein if the sex is male, said variable value is 1, if the relationship status is positive, said variable value is 1, if the completion status of intake form is completed, said variable value is 1, if the mood disorder diagnosis is positive, said variable value is 1, if the anxiety disorder diagnosis is positive, said variable value is 1, if the attention disorder diagnosis is positive, said variable value is 1, if the pre-visit status is positive, said variable value is 1, and if the provider physician status is positive, said variable value is 1.

15. The computer-implemented method of claim 8, wherein, for the one or more continuous variables, outliers are removed using a kernel density estimation approach.

16. The computer-implemented method of claim 8, wherein, for the one or more continuous variables, outliers are removed by removing variables having a probability density lower than a predetermined percentage of a maximum density.

17. The computer-implemented method of claim 1, wherein the plurality of trials includes all permutations for the one or more models and the one or more parameter configurations.

18. A non-transitory computer readable medium having a set of instructions stored thereon that, when executed by a processing device, cause the processing device to carry out an operation of treatment adherence prediction, the operation comprising:
receiving a full dataset;
isolating a training dataset and a test dataset from the full dataset;
splitting the training dataset into one or more training folds and a validation fold;
training, over a plurality of trials, one or more models on each of the one or more training folds for each of one or more parameter configurations correlated to said one or more models, each of the one or more models configured to classify a target variable,
wherein the target variable is an indicator of whether a patient will complete a treatment;
validating, for each of the one or more models for a given trial of the plurality of trials, a given model of the one or more models on the validation fold;
comparing a training score with a validation score,
wherein the training score is based on the training of the one or more models over the one or more training folds, and
wherein the validation score is based on the validating of the given model on the validation fold;
recording classifications scores for each of the one or more models, the classifications scores based on the training score and the validation score for each of the one or more models;
selecting a selected model from the one or more models, the selected model having a top classification score;
retraining the selected model on the full dataset; and
generating adherence predictions for the test dataset.

19. A system for treatment adherence prediction, the system comprising a server comprising at least one server processor, at least one server database, at least one server memory comprising a set of computer-executable server instructions which, when executed by the at least one server processor, cause the server to:
receive a full dataset;
isolate a training dataset and a test dataset from the full dataset;
split the training dataset into one or more training folds and a validation fold;
train, over a plurality of trials, one or more models on each of the one or more training folds for each of one or more parameter configurations correlated to said one or more models, each of the one or more models configured to classify a target variable,
wherein the target variable is an indicator of whether a patient will complete a treatment;
validate, for each of the one or more models for a given trial of the plurality of trials, a given model of the one or more models on the validation fold;
compare a training score with a validation score,
wherein the training score is based on the training of the one or more models over the one or more training folds, and
wherein the validation score is based on the validating of the given model on the validation fold;
record classifications scores for each of the one or more models, the classifications scores based on the training score and the validation score for each of the one or more models;
select a selected model from the one or more models, the selected model having a top classification score;
retrain the selected model on the full dataset; and
generate adherence predictions for the test dataset.
